(19) United States
(12) Patent Application Publication
(10) Pub. No.: US 2024/0285285 A1
(43) Pub. Date: Aug. 29, 2024

(54) THREE JAW CLAMP APPARATUS AND SYSTEM FOR OCCLUSION AND EVERSION OF BLOOD VESSEL AND METHOD THEREOF

(71) Applicant: SHIRA MEDTECH PRIVATE LIMITED, Ahmedabad (IN)

(72) Inventors: Anand PARIKH, Ahmedabad (IN); Prasann JAIN, Udaipur (IN); Aakash KOHLI, Lucknow (IN)

(21) Appl. No.: 18/573,444

(22) PCT Filed: Jun. 13, 2022
(86) PCT No.: PCT/IB2022/055430

§ 371 Date: Dec. 22, 2023

(30) Foreign Application Priority Data
Jun. 25, 2021 (IN) 202121028590

(51) Int. Cl. A61B 17/122; A61B 17/11; A61B 17/12; A61B 17/30

(52) U.S. Cl. CPC A61B 17/1227; A61B 2017/1107; A61B 2017/12004; A61B 2017/301

(57) ABSTRACT

Exemplary embodiments of the present disclosure are directed towards a three jaw clamp apparatus and system for occlusion and eversion of blood vessel and method thereof. The present disclosure provides an apparatus for occluding and opening a blood vessel. The apparatus is a three jaw design, for occluding and opening the blood vessel while preventing excessive stretching of adventitia or tearing of the blood vessel/adventitia. The three jaw clamps provide reliable clamps and can be cleaned easily and includes a first member, a second member, and a third member for occluding and opening the blood vessel that can be joined/assembled/welded fast. The system for occlusion, inversion and approximation of two ends of blood vessels while preventing excessive stretching of adventitia or tearing of the blood vessel/adventitia. The system of three jaw clamps can adjust the distance between the two ends to facilitate anastomosis.

### TECHNICAL FIELD

The disclosed subject matter relates generally to the field of anastomosis and anastomotic devices. Particularly, the present disclosure relates to a three jaw clamp apparatus and system for occlusion and eversion of blood vessel and method thereof.

### BACKGROUND

Vascular anastomosis is a surgical procedure for connecting two blood vessels. The vascular anastomosis is a crucial procedure in plastic and reconstructive surgeries, microsurgical techniques/microsurgeries in humans and also in veterinary surgeries of small and large animals. Vascular anastomosis may include but not be limited to surgical vascular anastomosis such as peripheral vascular, renal, brain, carotid artery, etc. Secondarily, it can be applied to veterinary surgeries of small and large animals.

A microsurgical transfer/microsurgery in the context of a double vascular anastomosis refers to less than 3 mm distance between vessels in the transferred tissue to the ones in the recipient area. This may also include scenarios in which anastomoses have a diameter between 0.3 and 0.8 mm The standard procedure to perform vascular anastomosis is to first clamp the blood vessels of interest using clamps in order to prevent blood flowing into the surgical site. This is of utmost importance in order to ascertain the efficiency and accuracy of the microsurgery. The standard procedure involves removal of the tunica-adventitia (outermost layer of the blood vessels) thereby preventing it from a) interfering with anastomosis, and b) herniating into the lumen thus causing formation of thrombus, and to facilitate the application of nylon sutures to connect the clamped vessel ends. Application of the clamp usually leads to the collapse of the lumen and in veins causes the closure of the end of the vessels. The process of anastomosis requires a very specific set of skills to suture the ends while restraining the ends from collapsing. The entire procedure of micro-vascular anastomosis is typically carried out under loupes or a microscope. However, there are certain disadvantages associated with the use of such conventional clamps. The use of conventional clamps collapses the vessel lumen and causes the closure of the end vessels of veins. This in turn restricts the view of the opening and closing end of the vessel being operated by surgeon.

Another conventional surgical methodology includes the use of Acland clamp. The standard for temporary occlusion of the micro-vessels during the anastomosis requires employing an Acland clamp, the design of which has more or less remained unchanged since its inception nearly three decades ago. Sutures of suitable size are used in combination with Acland clamps, for anastomosing the occluded vessels. Despite using Acland clamps in the procedure, drawback of vessel collapsing remains. There is no device which prevents the vessels from collapsing or aids in opening the lumen of the collapsed vessel after the application of the Acland clamp. For opening the lumen of the collapsed blood-vessel walls post occlusion, an assistant to the surgeon constantly irrigates the vessel lumen with heparinized saline solution. A pair of the Acland clamps (approximator clamps) are typically used which have either straight or angulated jaws. In additional, framed approximator clamps are used as well. These clamps are usually made of a stainless steel or a polycarbonate material. These surgical devices are operated using clamp applying forceps, which have designs with a locking mechanism, making them complex to use and operate.

Yet another conventional system uses a device named Unilink coupler that was first introduced by Ostrup and Olding in 1976. Today after several improvements, it is known as 3M precise anastomotic device, which is used for end-to-end venous anastomoses for free-flap transfers. It uses titanium links/staples as a fastening element between two ends of the vessels to be connected. With regard to surgical performance, the clipped microvascular anastomosis proved to be significantly faster and more ergonomic. The Unilink coupler is used in conjunction with the Acland clamp. However, its application is limited only to end-to-end venous anastomosis. It cannot be used on arteries and also in other common procedures such as end-to-side anastomosis, sleeve anastomosis and so on.

Although, state of art in this technical field is replete with conventional surgical interventions for vascular anastomosis, there are many caveats in using conventional clamps and other accessory devices for vascular anastomosis. Some of the demerits include excessive stretching of adventitia, resulting in either damaging the blood vessel or tearing of adventitia from blood vessel, rendering it useless, causing collapse the vessel lumen and closure of the end vessels of veins thereby restricting the surgical view of the opening and closing end of the vessel during the surgical procedure, increased strains on the micro components comprising the conventional clamps reduce the durability and decrease the process efficiency in general, failure to provide a non-disposable and a reusable device, increased number of steps to assemble the device, inability to provide efficacious cleaning provisions due to complicated designs. The conventional clamp fails to provide an apparatus and system for occlusion and eversion blood vessel which reduces maximum stress on the clamp thus hampering the device reliability. In most of devices flipping the clamp occurs, consequently are not convenient for making micro-anastomosis in space constrained surgical sites such as head and neck. Further, if the blood vessel to be occluded is far from device's end and user tries to hold adventitia then, lumen (opening) of the blood vessel will face away from the surgeon and not towards the surgeon. Last but not the least, most conventional surgical procedures incorporating the use of conventional devices require skilled assistance.

In light of the aforementioned discussion, there exists a need for a system with novel methodologies that would overcome the above-mentioned disadvantages. Further, state of art fails to suggest a technique of holding adventitia or the number of jaws, and cannot be directly applied to a occluding and opening of blood vessels. Although, conventional devices can be presented as suited to the purposes for which they were projected, they are not so suited for the purposes of the present invention, as described herein below.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An objective of the present disclosure is directed towards a three jaw clamp apparatus and system for occlusion and eversion of blood vessel and method thereof.

Another objective of the present disclosure is directed towards a three jaw clamp apparatus and system for occlusion and eversion of blood vessel to prevent excessive stretching of adventitia, thereby averting the blood vessel damage.

Another objective of the present disclosure is directed towards a three jaw clamp apparatus and system for occlusion and eversion of blood vessel that has several improvements and technical advancements thereby increasing reliability of the said device when compared to conventional devices.

Another objective of the present disclosure is directed towards a three jaw clamp apparatus and system for occlusion and eversion of blood vessel that restricts inadvertent motion of the device in a system of two clamps.

Another objective of the present disclosure is directed towards a three jaw clamp apparatus and system for occlusion and eversion of blood vessel that makes cleaning of the device easier than predecessor devices.

Another objective of the present disclosure is directed towards a three jaw clamp apparatus and system for occlusion and eversion of blood vessel that reduces number of steps to join/weld/assemble the device thereby providing an ergonomic design with uncomplicated usage steps and increased procedural efficiency.

Another objective of the present disclosure is directed towards a cost-effective, simplified device that improves the procedural efficiency, reduces the chance of errors and enables surgery to take place in an easy manner without the need for accessory devices.

Yet another objective of present disclosure is to provide a three jaw clamp apparatus and system with lower stresses on clamp reducing the probability of reduction in pinching force increasing the reliability of the device.

Yet another objective of present disclosure is to control the stretching of adventitia thereby reducing the chances of causing trauma.

Yet another objective of present disclosure is to provide a three jaw clamp apparatus and system for occlusion and eversion blood vessel with evenly distributed stress level on the clamp increasing the reliability.

Yet another objective of present disclosure is to provide a non-disposable and reusable apparatus for occlusion and eversion blood vessel.

Yet another objective of present disclosure is to reduce the number of steps required in assembling/joining/welding of the device.

Yet another objective of present disclosure is to provide a three jaw clamp apparatus and system for occlusion and eversion blood vessel with increased length of the spring in the clamp reducing stress on biasing member of the device.

Yet another objective of present disclosure is to provide an apparatus and system for occlusion and eversion blood vessel with three jaws.

Yet another objective of present disclosure is to provide a three jaw clamp apparatus and system for occlusion and eversion blood vessel that eliminates the chances of erroneous posterior wall suture (back-walling) due to better visibility of the posterior wall.

Yet another objective of present disclosure is to eliminate the need for flipping the clamp, thus making micro-anastomosis more convenient in space constrained surgical sites such as head and neck.

Yet another objective of present disclosure is to reduce the requirement of skilled assistance during surgery.

Yet another objective of present disclosure is to provide a three jaw clamp apparatus and system that may prove extremely beneficial in surgical procedures other than blood vessels, such as surgical procedures involving vessels having tubular structures such as vas-deferens (for vasectomy reversal) etc.

Yet another objective of the present disclosure is to provide a three jaw clamp apparatus and system for occlusion and eversion of blood vessel and method thereof. The three jaw microvascular clamps described herein to keep the blood vessel walls from collapsing after occlusion. With this functionality, these clamps offer the following clinical advantages namely: 1. Eliminates chances of erroneous posterior wall suture (back-walling) due to better visibility of the posterior wall. 2. No need for flipping the clamp, thus making micro-anastomosis more convenient in space constrained surgical sites such as head and neck and 3. Reduces the requirement of skilled assistance during surgery

Another objective of the present disclosure is directed towards a three jaw clamp apparatus for occluding a blood vessel and opening lumen of the blood vessel, the three jaw clamp apparatus comprising: a first member, a second member, and a third member; wherein each the member comprises a handle and a jaw; and wherein the first member and the second member being laterally moveable toward and away from one another; wherein the third member being laterally moveable toward and away from the first member; wherein the first member and the second member occlude the blood vessel in between corresponding jaws when a pinch force is applied on the handle of the first member and the handle of the second member comprising: applying the pinch force, on the handle of the first member and the handle of the second member, causing corresponding jaws to open; positioning the open jaws about the blood vessel; and releasing the pinch force, on the handle of the first member and the handle of the second member, causing the open jaws to close on the blood vessel; wherein the third member and the first member occlude an adventitia of the blood vessel in between corresponding jaws when a pinch force is applied on the handle of the third member and the handle of the first member comprising: applying the pinch force, on the handle of the third member and the handle of the first member, causing corresponding jaws to open; positioning the adventitia between the open jaws; and releasing the pinch force, on the handle of the third member and the handle of the first member, causing the open jaws to close on the adventitia; and wherein the third member and the first member maintain a tension in the adventitia by creating a holding tension on the adventitia to open the blood vessel, wherein the jaw of the third member having width less than 60% of that of the jaw of the first member, and wherein the distance between tip of the jaw of the third member and a root of the adventitia is less than quarter of the perimeter of maximum size blood vessel to be occluded by the three jaw clamp apparatus.

Other features and additional objectives of the present invention will become apparent from the following descriptions. These features will be described at sufficiently detailed levels to allow the technicians of the subject matter to implement the invention. Also, it is understood that other features can be used and structural changes can be made without leaving the scope of the invention. In the accompanying drawings, like reference numbers indicate identical or like parts throughout the several views. Therefore, the following detailed description should not be taken as limiting the scope of the present invention which is defined by the appended claims.

### BRIEF DESCRIPTION OF THE DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 represented by 100 is a side view of the present disclosure, in accordance with one or more exemplary embodiments.

FIG. 2 represented by 200 is a perspective view of FIG. 1 of the present disclosure, in accordance with one or more exemplary embodiments.

FIG. 3 represented by 300 is a perspective view of member 1 in three jaw clamp 100 of the present disclosure, in accordance with one or more exemplary embodiments.

FIG. 4 represented by 400 contains images that explains step by step procedure to hold adventitia using the present disclosure, in accordance with one or more exemplary embodiments.

FIG. 5 represented by 500 contains table of standard dimensions of member 1 that are used across the industry for different sizes of blood vessel, in accordance with one or more exemplary embodiments.

FIG. 6 represented by 600 is another perspective of the present disclosure that increases reliability of the device, in accordance with one or more exemplary embodiments.

FIG. 7 represented by 700 is another perspective of the present disclosure that increases reliability of the device, in accordance with one or more exemplary embodiments.

FIG. 8 represented by 800 is another perspective of the present disclosure that increases reliability of the device, in accordance with one or more exemplary embodiments.

FIG. 9 represented by 900 is another perspective of the present disclosure that increases reliability of the device, in accordance with one or more exemplary embodiments.

FIG. 10 represented by 1000 is another perspective of the present disclosure that increases reliability of the device, in accordance with one or more exemplary embodiments.

FIG. 11 represented by 1100 is another perspective of the present disclosure that increases reliability of the device, in accordance with one or more exemplary embodiments.

FIG. 12 represented by 1200 is another perspective of the present disclosure that makes cleaning of the device easy, in accordance with one or more exemplary embodiments.

FIG. 13 represented by 1300 is another perspective of the present disclosure that reduces the number of steps required in assembly/joining/welding of the device, in accordance with one or more exemplary embodiments.

FIG. 14 represented by 1400 is another perspective of the present disclosure that increases reliability of the device, in accordance with one or more exemplary embodiments.

FIG. 15 represented by 1500 is a perspective view of FIG. 16, in accordance with one or more exemplary embodiments.

FIG. 16 represented by 1600 is a side perspective view of the present disclosure comprising a system of two clamps, in accordance with one or more exemplary embodiments.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and so forth, herein do not denote any order, quantity, or importance, but rather are used to distinguish one element from another.

The present disclosure provides an apparatus and system for occlusion and eversion blood vessel. More pertinently, the present disclosure relates to an apparatus and system for occlusion and eversion blood vessel which reduces the stress on the clamp. The apparatus is a three jaw design, for occluding and opening the blood vessel while preventing excessive stretching of adventitia or tearing of the blood vessel/adventitia. The three jaw clamps provide reliable clamps and can be cleaned easily. The three jaw clamps provide includes a first member, a second member, and a third member. The three jaw clamps for occluding and opening the blood vessel can be joined/assembled/welded fast. Further, the present disclosure provides a system for occlusion, inversion and approximation of two ends of blood vessels while preventing excessive stretching of adventitia or tearing of the blood vessel/adventitia. The system of three jaw clamps can adjust the distance between the two ends to facilitate anastomosis. The system doesn't experience inadvertent motion of any component in the assembly. The present disclosure provide a mechanism for maintaining tension on the adventitia to open the blood vessel while preventing excessive stretching of adventitia or tearing of the blood vessel/adventitia.

In an embodiment, the apparatus includes three jaw design clamp with varying in its dimension and orientation. The three jaw clamps consist of member, each includes a handle and a jaw. The apparatus includes a first member, a second member, and a third member in the three jaw clamps. The first member and the second member are laterally moveable towards and away from one another. The third member is laterally moveable towards and away from the first member. The first member and the second member occludes the blood vessel in between corresponding jaws when a pinch force is applied on the handle of the first member and the handle of the second member. The third member and the first member occludes adventitia of the blood vessel in between corresponding jaws when a pinch force is applied on the handle of the third member and the handle of the first member or the second member. The third member and the first member maintain the tension in the adventitia by creating a holding tension on the adventitia to open the blood vessel.

Another embodiment of the present disclosure provides a system of microvascular clamps with retractor unit for occluding the blood vessel and retracting its end. It provides a system for occlusion of blood vessel and retraction of blood vessel ends. The system is such that it adjusts the distance between the two ends to facilitate anastomosis. Moreover the three jaw clamp with retractor system does not experience inadvertent motion of any component in the assembly. The retractor in three jaw clamps member facilitates uniform motion and increases friction. Present embodiment of three jaw clamps with retractor facilitates the blood vessel to open its ends with/without using adventitia, thereby eliminating the dependency on adventitia of the blood vessel. Retracting the blood vessel makes posterior wall visible to the surgeon while doing anastomosis. Thus, allowing surgeon to start suturing posterior wall first and then the anterior wall without flipping the clamp. This prevents twisting of vessels and thereby reduces chances of trauma that can occur to them.

In another embodiment of the present disclosure, a system of microvascular clamps with three jaw clamps includes two clamps that are assembled together with an approximator rod. Approximator rod can be of any cross section. Its functionality allows three jaw clamps to slide towards and away from each other, so that surgeon can adjust the ends of blood vessel as per their requirement. Ends of approximator rod have a larger/different cross section than the middle portion. This is to prevent clamps or any other component of the system of two clamps from getting detached from the system of two clamps.

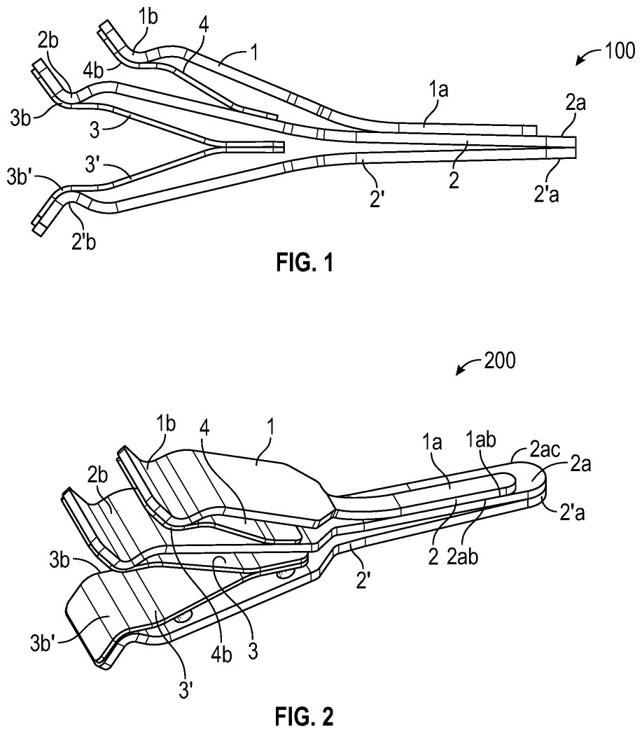

Referring to FIG. 1 represented by 100 is a side view of a three jaw clamp 100. The Three jaw clamp 100 consist of 6 members. Member 1, 2 and 2′ consist of jaws 1a, 2a and 2′a and handles 1b, 2b and 2′b. Member 1, 2 and 2′ are laterally moveable toward and away from each other. Member 2′ is laterally moveable toward and away from the member 2. Member 1 is laterally moveable toward and away from member 2. Member 2 and 2′ occludes the blood vessel between their jaws 2a and 2′a when pinching force is released from the handle 2b and 2′b. Member 1 and 2 holds adventitia between their jaws 1a and 2a when pinching force is released from the handle 1b and 2b. Three jaw clamp 100 includes at least one biasing unit 4 is placed between 1b and 2b to impart the pinching force along the length of jaws 1a and 2a. Similarly, in clamp 100 at least one biasing unit (3 & 3′) placed between 2b and 2′b to impart the pinching force along the length of jaws 2a and 2′a. Handle 1b is designed and assembled with biasing member 4 in the clamp 100 such that portion of 4b that is nearest to surface A of member 2 touches the surface A of member 2 when member 4 is in its maximum stress condition while in function. Similarly, handles 2b and 2′b comprise portions 3b and 3′b respectively.

Referring to FIG. 2 is a perspective view of FIG. 1 (100), in accordance with one or more exemplary embodiments.

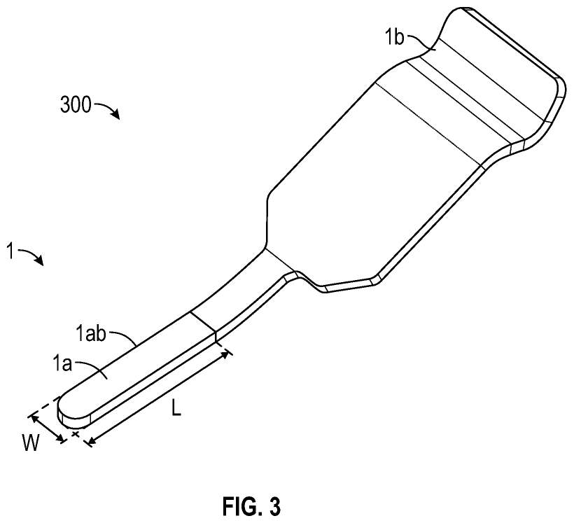

FIG. 3 is a perspective view of member 1 of three jaw clamp 100, in accordance with one or more exemplary embodiments. Jaw 1a of member 1 of clamp 100, is designed and assembled with the clamp 100 in such a way that it prevents excessive stretching of adventitia. Edge lab of jaw 1a should be as near as possible to the edge 2ab (shown in FIG. 2) of jaw 2a. In the present disclosure when a pinch force is applied, by the operator, on the handle 2b and 2′b, so as to cause to open jaw 2a relative to 2′a. The open jaws 2a and 2′a are positioned about the blood vessel. The pinch force is released on the handle 2b of member 2 and 2′b of member 2′ so as to cause the open jaws 2a and 2′a to close on the blood vessel. Member 2 and 2′ occlude the blood vessel in between corresponding jaws 2a and 2′a when the pinch force is applied by the biasing unit 4, on the handle 2b of member 2 and handle 2′b of member 2′. In the present disclosure when a pinch force is applied, by the operator, on the handle 2b and 1b, so as to cause to open jaw 2a relative to 1a. Adventitia is positioned between jaws 2a and 1a using a forceps. The pinch force is released on the handle 2b of member 2 and 1b of member 1 so as to cause the open jaws 2a and 1a to close on the blood vessel. Member 2 and 1 occlude the blood vessel in between corresponding jaws 2a and 1a when the pinch force is applied by the biasing unit 4, on the handle 2b of member 2 and handle 1b of member 1. FIG. 3 also represents W and L as width and length dimensions respectively.

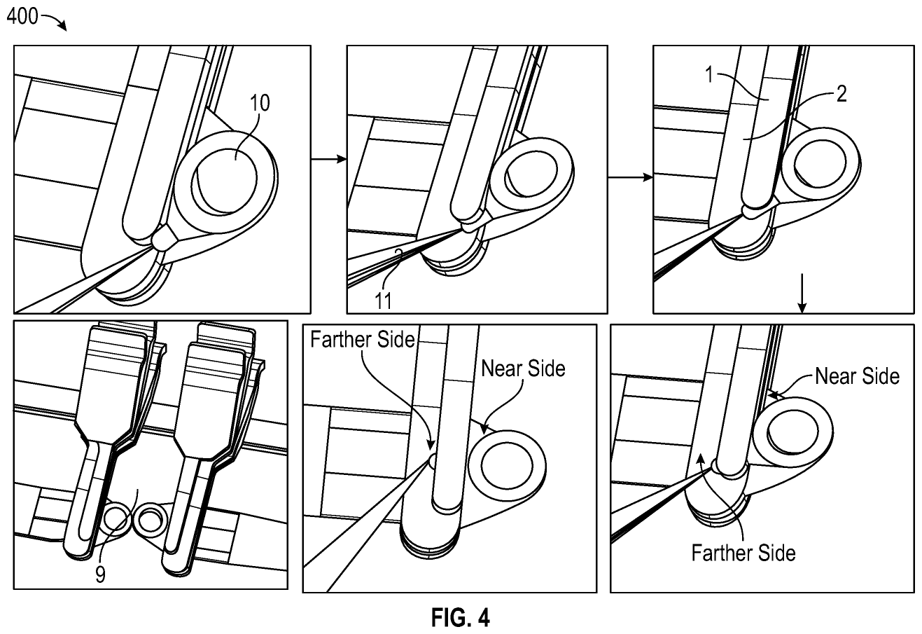

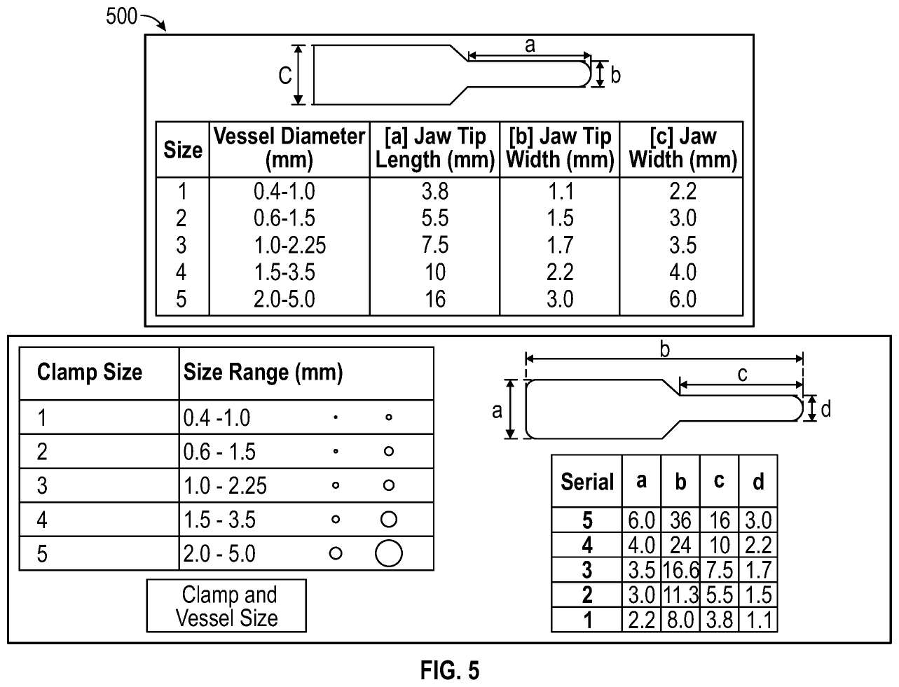

FIG. 4 (400) shows a step by step procedure of holding the adventitia, in accordance with one or more exemplary embodiments. Adventitia is grabbed and stretched to tip of jaw 1a as shown in image 1, 2 and 3 of FIG. 4. Since, very small length of adventitia is available; too much stretching can result in damaging the blood vessel or tearing of adventitia from blood vessel, rendering it useless. This poses a constraint on jaw length of adventitia holding jaw of a three jaw design. If jaw length is very high then adventitia will be stretched to a length that can tear it out from blood vessel. Thus, minimum distance between tip of jaw 1a and the root of the adventitia (If the topmost adventitia is held, then the root of the adventitia will be vertically above the axis of occluded blood vessel) should be less than quarter of the perimeter of maximum size blood vessel that is advised to be occluded by the device. In the next step, adventitia is stretched around the jaw tip of member 1 to reach its farther side as shown in image 4 and 5 of below figure. This poses a constraint on jaw width of member 1. If jaw width is very high then adventitia will be stretched to a length that can tear it out from blood vessel. Width “W” of jaw 1a in the present disclosure should be less than the natural length of adventitia that is available for stretching. Standard clamp sizes that are currently used for different vessel sizes in the industry are given in FIG. 5 Following standard lengths given in FIG. 5, width “W” of jaw 1a should be less than 60% of the diameter of maximum size blood vessel that is advised to be occluded using the device, in accordance with one or more exemplary embodiments. FIG. 4 includes reference numerals 9, 10 and 11

FIG. 5 (500) contains table of standard dimensions of member 1 that are used across the industry for different sizes of blood vessel, in accordance with one or more exemplary embodiments.

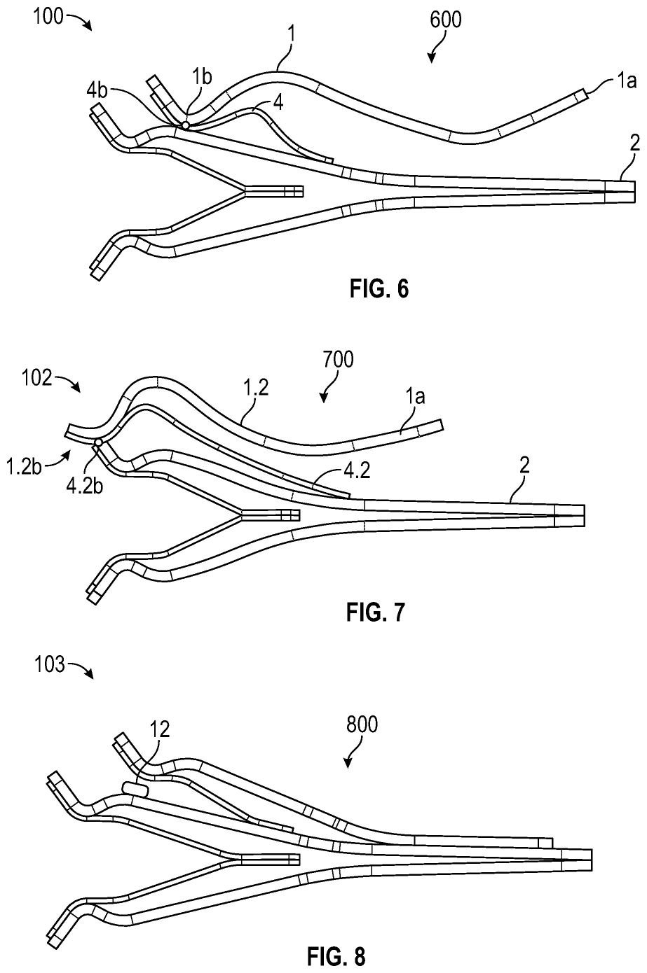

FIG. 6 (600) is another perspective of the present disclosure that increases reliability of the device, in accordance with one or more exemplary embodiments.

FIG. 7 (700) is a side view of three jaw clamp 102, in accordance with one or more exemplary embodiments. Keeping overall functionality of the clamp 102 same as clamp 100, if member 1 and 4 of three jaw clamp 100 are replaced with member 1.2 and 4.2 respectively then the result is three jaw clamp 102. If handles 1b and 4b are replaced with 1.2b and 4.2b respectively then the result is member 1.2 and 4.2 respectively. Handle 1.2b and 4.2b are designed and assembled in the clamp 102 such that portion of 4.2b nearest to the end of handle 2b touches the end of handle 2b when member 4.2 is in its maximum stress condition while in function. Member 1.2 comprises a jaw 1.2a and a handle 1.2b. Member 1.2 is laterally moveable toward and away from member 2. Member 1.2 and 2 holds adventitia between their jaws 1.2a and 2a when pinching force is released from the handle 1.2b and 2b. Three jaw clamp 102 includes at least one biasing unit 4.2 placed between 1.2b and 2b to impart the pinching force along the length of jaws 1a and 2a. This design reduces deformation of biasing member 4.2, thereby reducing the stresses experienced by it. This increases reliability of the clamp.

FIG. 8 (800) is a side view of three jaw clamp 103, in accordance with one or more exemplary embodiments. Keeping overall functionality of the clamp 103 same as clamp 100, stopper 12 is assembled/joined/welded to handle 2a of member 2 of clamp 100 such that it restricts the maximum deformation of member 1 and member 4 when a pinching force is applied on handle 1b of member 1. This design reduces deformation of biasing member 4 in clamp 100, thereby reducing the stresses experienced by it. This increases reliability of the clamp. This can be used in conjunction with the clamp 104, 105, 106, 108 and 101. It may be noted that clamps described herein may be used as stand alone or in conjunction with each other.

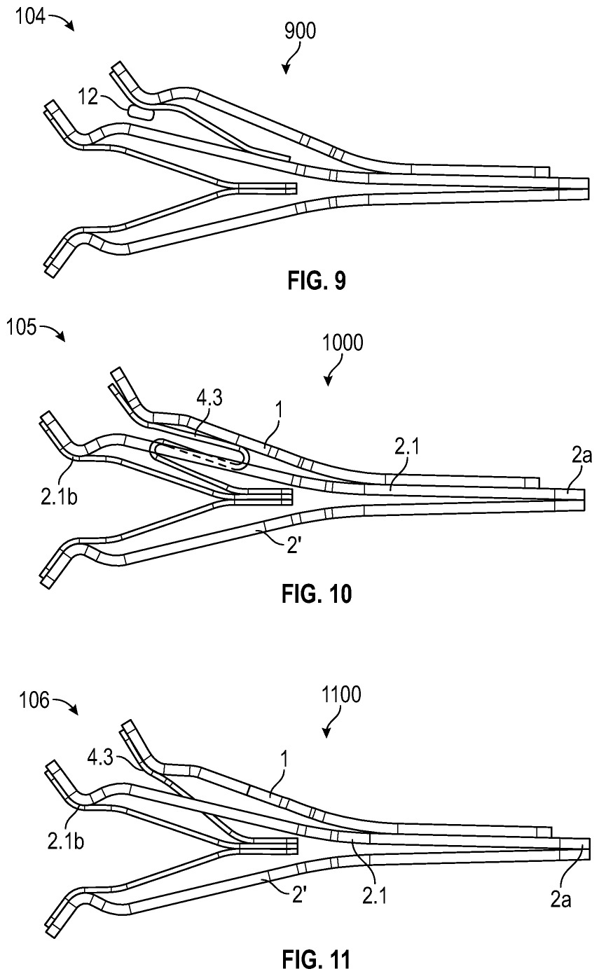

FIG. 9 (900) is a side view of three jaw clamp 104, in accordance with one or more exemplary embodiments. Keeping overall functionality of the clamp 104 same as clamp 100, stopper 12 is assembled/joined/welded to handle 4a of member 4 of clamp 100 such that it restricts the maximum deformation of member 1 and member 4 when a pinching force is applied on handle 1b of member 1. This design reduces deformation of biasing member 4 in clamp 100, thereby reducing the stresses experienced by it. This increases reliability of the clamp. This can be used in conjunction with clamp 103, 105, 106, 108 and 101.

FIG. 10 (1000) is a side view of three jaw clamp 105, in accordance with one or more exemplary embodiments. Keeping overall functionality of the clamp 105 same as clamp 100, if biasing member 4 and member 2 of clamp 100 is replaced by 4.3 and 2.1 then the result is three jaw clamp 105. If handle 2b is replaced with 2.1b then the result is member 2.1. Handle 2.1b of member 2.1 has a cavity in it such that one end of biasing member 4.3 can be joined/welded/integral part of biasing member 3 while other end 4.3b is joined/welded/integral part of handle 1b of member 1. Further biasing member 4.3 has a zigzag design. While in function, this design of biasing member 4.3 experiences less stresses as compared to the biasing member 4 of clamp 100 by distributing the stresses more evenly. Lower stresses increases reliability of the clamp. This can be used in conjunction with clamp 102.

FIG. 11 (1100) is a side view of three jaw clamp 106, in accordance with one or more exemplary embodiments. Keeping overall functionality of the clamp 106 same as clamp 100, if biasing member 4 and member 2 of clamp 100 is replaced by 4.3 and 2.1 then the result is three jaw clamp 106. Handle 2.1b of member 2.1 has a cavity in it such that one end of biasing member 4.4 can be joined/welded/integral part of biasing member 3 while other end 4.4b is joined/welded/integral part of handle 1.3b of member 1.3. In this configuration, length of member 4.4 of clamp 106 is more than member 4 of clamp 100. With bigger length, to achieve same amount of deflection, lower force is required and it also lead to lower maximum stress. Therefore, while in function, stresses experienced by biasing member 4.4 of clamp 106 is less than member 4 of clamp 100. Lower stresses increasing the reliability of the apparatus and it may be used in conjunction with clamp 102.

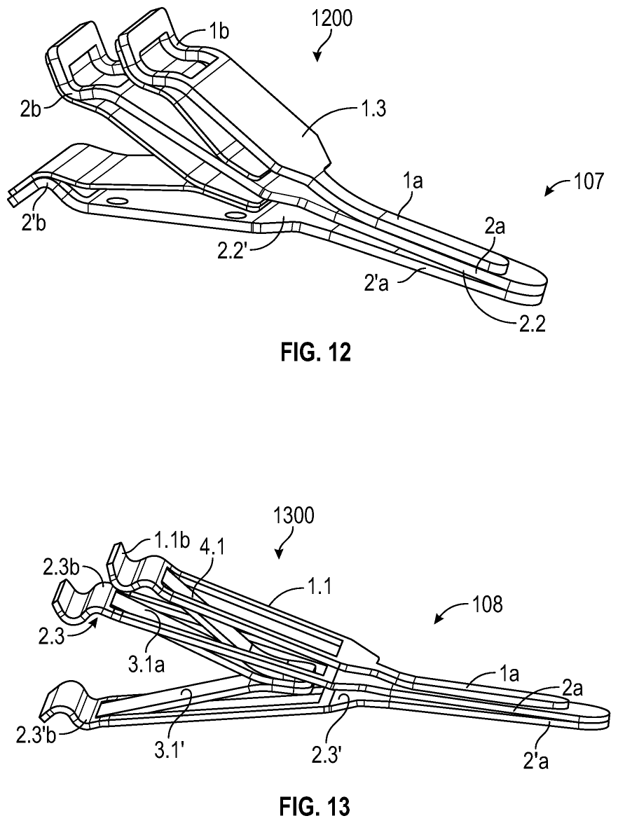

FIG. 12 (1200) is a perspective view of three jaw clamp 107, in accordance with one or more exemplary embodiments. Keeping overall functionality of the clamp 104 same as clamp 100, if member 1, 2 and 2′ in clamp 100 are replaced with 1.3, 2.2 and 2.2′ respectively then the result is three jaw clamp 107. If handle 1b, 2b and 2′b are replaced with 1.3b, 2.2b and 2.2′b respectively then the result is member 1.3, 2.2 and 2.2′ respectively. Handle 1.3b, 2.2b and 2.2′b have cavities near the location where they are joined/welded with handle 4b, 3b and 3′b respectively. Cavities can help in reaching obscure crevices/places in the clamp. After the usage clamp, 107 can be cleaned easily and more effectively than clamp 100. This can be used in conjunction with the clamp 103, 104, 105, 106 and 101.

FIG. 13 (1300) is a perspective view of three jaw clamp 108, in accordance with one or more exemplary embodiments. Keeping overall functionality of the clamp 108 same as clamp 100, if member 1 and 4 of clamp 100 are replaced by a single member 1.1 and member 2 and 3 of clamp 100 are replaced by a single member 2.3 and 2′ and 3′ of clamp 100 are replaced by a single member 2.3′ then clamp 108 is formed. If handle 1b, 2b and 2′b of clamp 100 is replaced by handle 1.1b, 2.3b and 2.3′b respectively then member 1.1, 2.3 and 2.3′ are formed. Biasing member 4.1, 3.1 and 3.1′ are integrated with handle 1.1b, 2.3b and 2.3′b respectively. Member 1.1, 2.3 and 2.3′ are laterally moveable toward and away from each other. Member 2.3′ is laterally moveable toward and away from the member 2.3. Member 1.1 is laterally moveable toward and away from member 2.3. Member 2.3 and 2.3′ occludes the blood vessel between their jaws 2a and 2′a when pinching force is released from the handle 2.3b and 2.3′b. Member 1.1 and 2.3 holds adventitia between their jaws 1a and 2a when pinching force is released from the handle 1.1b and 2.3b. Three jaw clamp 108 includes at least one biasing unit 4.1 integrated with handle 1.1b to impart the pinching force along the length of jaws 1a and 2a. Similarly, in clamp 100 at least one biasing unit integrated with handles 2b and 2′b to impart the pinching force along the length of jaws 2a and 2′a. Joining all members of the device is a time consuming task. If two or members are manufactured as a single member then step of joining them can be avoided Less joining locations mean less obscure places/crevices to reach. This design can be assembled/joined/welded fast and cleaned easily. This can be used in conjunction with clamp 102.

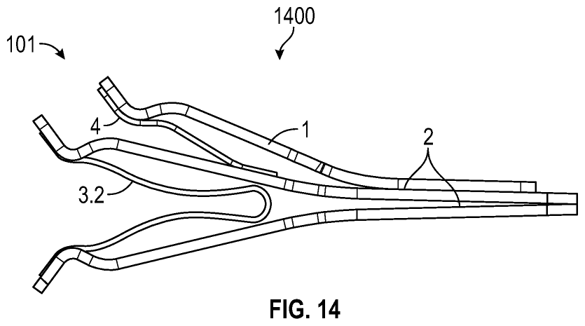

FIG. 14 (1400) is a side view of three jaw clamp 101, in accordance with one or more exemplary embodiments. Keeping overall functionality of the clamp 101 same as clamp 100, if biasing member 3 and 3′ of clamp 100 are replaced by a single member 3.2 then the result is three jaw clamp 106. This new member has higher radius at high stress regions. This helps in reducing the maximum stress levels experienced by member 3.2 of clamp 101 as compared to member 3 and 3′ of clamp 100 and thereby increasing reliability of the clamp 101. This can be used in conjunction with clamp 105 and 106.

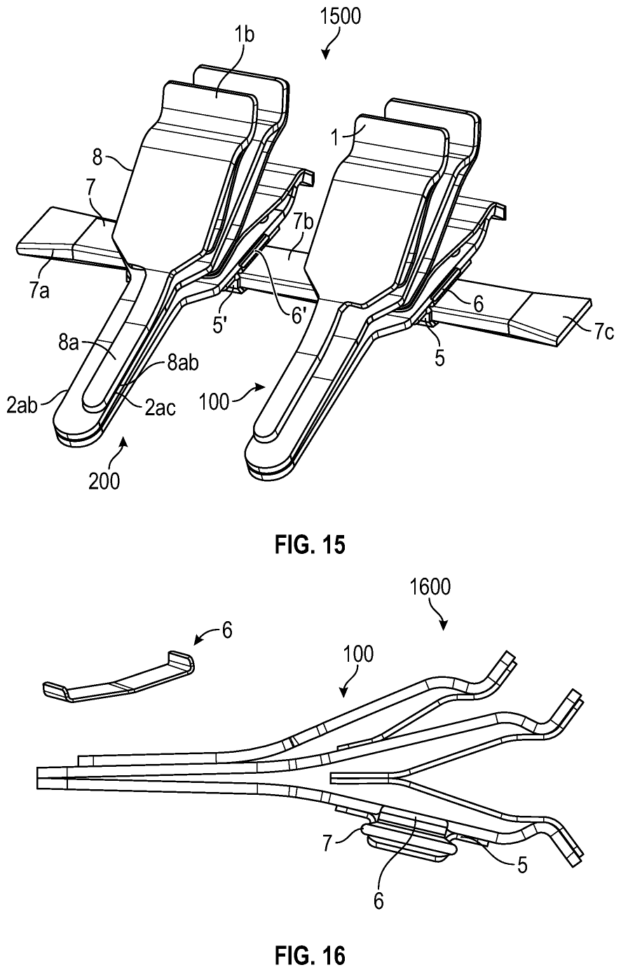

Referring to FIG. 15 (1500) is a side view of a system 1000 of two three jaw clamps 100 and 200, in accordance with one or more exemplary embodiments. If member 1 in jaw clamp 100 is replaced with member 8 then the result is three jaw clamp 200. If jaw 1a of member 1 is replaced with jaw 8a then the result is member 8. Edge 8ab of jaw 8a should be as close as possible to the edge 2ac of jaw 2a. Member 8 is a mirror image of member 1.

Both the three jaw clamps 100 and 200 are assembled together to form a system 1000. Member 5 and 5′ are joined/welded/integral part of member 2′ in clamp 100 and 200 respectively. Member 5 and 5′ are permanently joined to member 2′ (or could be an integrated part of member 2′) of clamp 100 and 200 respectively. While assembling system 1000, biasing member 6 and 6′ are placed in between member 7 and member 2′ of clamp 100 and 200 respectively. In system 1000, member 6 biases member 7 of system 1000 and member 2′ of clamp 100 against each other. Similarly, member 6′ biases member 7 of system 1000 and member 2′ of clamp 200 against each other. Member 6 and 6′ are designed such that they cannot inadvertently be detached from the system 1000. Member 7 has three parts 7a, 7b and 7c. In the system, assembly of clamp 100, member 5 and member 6 can slide over 7b part of member 7. Similarly, assembly of clamp 200, member 5′ and member 6′ can slide over member 7b part of member 7. Part 7a and 7c are wider than 7b. This prevents the assembly of clamp 100, member 5 and member 6 and the assembly of clamp 200, member 5′ and member 6′ to detach from the system 1000. The members 6 and 6′ force member 7 towards member 2′ of clamps 100 and 200 respectively. This results in generation of a contact. This contact generates a frictional force large enough to prevent undesired sliding of clamp 100 and 200 over member 7. This frictional force does not allow a relative motion between member 7 and the device unless sufficient external force is applied to deliberately cause relative motion. Moreover, the contact force restricts member 7 to move in the direction of force thus providing a smooth motion of device along member 7. Three jaw clamp present in the system 1000 can be of any type from 100 to 108 that has been described in the present disclosure.

Referring to FIG. 16 (1600) is a side aspect of the present disclosure that contains a system of two clamps, in accordance with one or more embodiments.

In an embodiment, a three jaw clamp apparatus for occluding a blood vessel and opening lumen of the blood vessel, the three jaw clamp apparatus comprising:
a first member, a second member, and a third member; wherein each the member comprises a handle and a jaw; and
wherein the first member and the second member being laterally moveable toward and away from one another;
wherein the third member being laterally moveable toward and away from the first member;
wherein the first member and the second member occlude the blood vessel in between corresponding jaws when a pinch force is applied on the handle of the first member and the handle of the second member comprising:
applying the pinch force, on the handle of the first member and the handle of the second member, causing corresponding jaws to open;
positioning the open jaws about the blood vessel; and releasing the pinch force, on the handle of the first member and the handle of the second member, causing the open jaws to close on the blood vessel;
wherein the third member and the first member occlude an adventitia of the blood vessel in between corresponding jaws when a pinch force is applied on the handle of the third member and the handle of the first member comprising:
applying the pinch force, on the handle of the third member and the handle of the first member, causing corresponding jaws to open;
positioning the adventitia between the open jaws; and
releasing the pinch force, on the handle of the third member and the handle of the first member, causing the open jaws to close on the adventitia; and
wherein the third member and the first member maintain a tension in the adventitia by creating a holding tension on the adventitia to open the blood vessel, wherein the jaw of the third member having width less than 60% of that of the jaw of the first member, and wherein the distance between tip of the jaw of the third member and a root of the adventitia is less than quarter of the perimeter of maximum size blood vessel to be occluded by the three jaw clamp apparatus.

In an embodiment, the jaw of the third member having width ranging between 0.6 mm-2.5 mm of that of the jaw of the first member, and wherein the distance between tip of the jaw of the third member and a root of the adventitia is less than quarter of the perimeter of maximum size blood vessel to be occluded by the three jaw clamp apparatus.

In an embodiment, the three jaw clamp apparatus comprises at least one biasing units placed between each adjacent pair of the handles of each the member to impart a pinch force along a length of corresponding jaws.

In an embodiment, wherein the tension in the adventitia in an outward direction opposes collapse of the blood vessel by lifting an intima of the blood vessel to open the blood vessel and keeps the blood vessel in an opened state.

In an embodiment, wherein the jaw of the first member and the jaw of the second member being selectively movable from a first position relative to the jaw of the first member and the jaw of the second member to a second position when the pinch force is applied on the handle of the first member and the handle of the second member.

In an embodiment, wherein the jaw of the first member and the jaw of the third member being selectively movable from a first position relative to the jaw of the first member and the jaw of the third member to a second position when the pinch force is applied on the handle of the first member and the handle of the third member.

In an embodiment, wherein the jaw of the first member and the jaw of the second member being selectively movable from a first position relative to the jaw of the first member and the jaw of the second member to a second position when the pinch force is applied on the handle of the first member and the handle of the second member.

In an embodiment, wherein the handles comprises at least one non-slip surface, at least one of a flat configuration, a curved configuration and combinations thereof.

In an embodiment, wherein the jaws comprises at least one non-slip surface, at least one of a flat configuration, a curved configuration and combinations thereof.

In an embodiment, the jaws comprise at least one insert mounted on at least one contact surface.

In an embodiment, the jaw and the handle are made of a single member and one or more joint members.

In an embodiment, the structure of the handle of third member is configured to travel a minimum distance before coming in contact to the handle of first member upon complete opening of the jaw of the third member relative to the jaw of the first member.

In an embodiment, an additional member placed between the handle of the first member and the handle of the third member is configured to reduce travel of the handle of the third member before coming in contact to the handle of the first member upon complete opening of the jaw of the third member relative to the jaw of the third member.

In an embodiment, the handles of the first, second and third member comprise structural gaps to permit the biasing unit(s) to pass through during their travel.

In an embodiment, the handles of the first, second and third member comprise structural gaps to permit cleaning agents and other accessory instruments to reach a space between the handles and biasing units.

In an embodiment, the handles of the first, second and third member comprise structural gaps to permit biasing units to pass through the structure of the handles of the members.

In an embodiment, members of biasing unit(s) are joined via welding at one point or surface, and handles of the first, second and third member comprise structural gaps to permit a welding electrode to reach the welding point or surface.

In an embodiment, the one or more biasing member(s) comprise a curved structure that reduces concentration of stress to promote longevity of the apparatus.

In an embodiment, the jaw of the third member is having width less than 60% of that of the jaw of the first member.

In an embodiment, a three jaw clamp system for occluding a blood vessel and opening a lumen of the blood vessel comprising:
a pair of three jaw clamp apparatus, wherein each of the three jaw clamp apparatus, occludes the blood vessels; and
a support member connecting the pair of three jaw clamp apparatus, in proximity to each other, wherein each of the three jaw clamp apparatus, comprises:
a first member, a second member, and a third member; wherein each the support member comprises a handle and a jaw; and
wherein the first member and the second member being laterally moveable toward and away from one another;
wherein the third member being laterally moveable toward and away from the first member;
wherein the first member and the second member occlude the blood vessel in between corresponding jaws when a pinch force is applied on the handle of the first member and the handle of the second member;
wherein the third member and the first member occlude an adventitia of the blood vessel in between corresponding jaws when a pinch force is applied on the handle of the third member and the handle of the first member; and
wherein the third member and the first member maintain a tension in the adventitia by creating a holding tension on the adventitia to open the blood vessel.
wherein the jaw of the third member having width less than 60% of that of the jaw of the first member, and wherein the distance between tip of the jaw of the third member and a root of the adventitia is less than quarter of the perimeter of maximum size blood vessel to be occluded by the three jaw clamp system.

In an embodiment, the jaw of the third member having width ranging between 0.6 mm-2.5 mm of that of the jaw of the first member, and wherein the distance between tip of the jaw of the third member and a root of the adventitia is less than quarter of the perimeter of maximum size blood vessel to be occluded by the three jaw clamp apparatus.

In an embodiment, the support member is one of a connector, an approximator, a retractor and a frame.

In an embodiment, a biasing member placed in contact between each approximator and a second member is configured to impart adequate frictional force large enough to prevent undesired sliding and provide smooth motion upon applying deliberate force to cause relative motion.

In an embodiment, the three jaw clamp present in the system 1000 can be of any type from 100 to 108 that has been described in the present disclosure. Alternatively, the three jaw clamps as disclosed herein may be used in conjunction with conventional clamps like Acland clamps and the use may include and not be limited to one or more units of three jaw clamps, one or more units of two jaw and three jaw clamps and any combinations thereof. The surgical procedures that the aforesaid combinations of surgical clamps would be used may include and not be limited to end-end anastomosis, end to side anastomosis, tubal vasectomy reversal and the like.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive. Thus the scope of the present disclosure is defined by the appended claims and includes both combinations and sub combinations of the various features described herein above as well as variations and modifications thereof, which would occur to persons skilled in the art upon reading the foregoing description.

We claim:

1. A three jaw clamp apparatus for occluding a blood vessel and opening lumen of the blood vessel, the three jaw clamp apparatus comprising:
a first member, a second member, and a third member; wherein each the member comprises a handle and a jaw; and
wherein the first member and the second member being laterally moveable toward and away from one another;
wherein the third member being laterally moveable toward and away from the first member;
wherein the first member and the second member occlude the blood vessel in between corresponding jaws when a pinch force is applied on the handle of the first member and the handle of the second member comprising:
applying the pinch force, on the handle of the first member and the handle of the second member, causing corresponding jaws to open;
positioning the open jaws about the blood vessel; and releasing the pinch force, on the handle of the first member and the handle of the second member, causing the open jaws to close on the blood vessel;
wherein the third member and the first member occlude an adventitia of the blood vessel in between corresponding jaws when a pinch force is applied on the handle of the third member and the handle of the first member comprising:
applying the pinch force, on the handle of the third member and the handle of the first member, causing corresponding jaws to open;
positioning the adventitia between the open jaws; and
releasing the pinch force, on the handle of the third member and the handle of the first member, causing the open jaws to close on the adventitia; and
wherein the third member and the first member maintain a tension in the adventitia by creating a holding tension on the adventitia to open the blood vessel, wherein the jaw of the third member having width less than 60% of that of the jaw of the first member, and wherein the distance between tip of the jaw of the third member and a root of the adventitia is less than quarter of the perimeter of maximum size blood vessel to be occluded by the three jaw clamp apparatus.

2. The three jaw clamp apparatus as claimed in claim 1, wherein the apparatus comprises at least one biasing units placed between each adjacent pair of the handles of each the member to impart a pinch force along a length of corresponding jaws.

3. The three jaw clamp apparatus as claimed in claim 1, wherein the tension in the adventitia in an outward direction opposes collapse of the blood vessel by lifting an intima of the blood vessel to open the blood vessel and keeps the blood vessel in an opened state.

4. The three jaw clamp apparatus as claimed in claim 1, wherein the jaw of the first member and the jaw of the second member being selectively movable from a first position relative to the jaw of the first member and the jaw of the second member to a second position when the pinch force is applied on the handle of the first member and the handle of the second member.

5. The three jaw clamp apparatus as claimed in claim 1, wherein the jaw of the first member and the jaw of the third member being selectively movable from a first position relative to the jaw of the first member and the jaw of the third member to a second position when the pinch force is applied on the handle of the first member and the handle of the third member.

6. The three jaw clamp apparatus as claimed in claim 1, wherein the jaw of the first member and the jaw of the second member being selectively movable from a first position relative to the jaw of the first member and the jaw of the second member to a second position when the pinch force is applied on the handle of the first member and the handle of the second member.

7. The three jaw clamp apparatus as claimed in claim 1, wherein the handles comprises at least one non-slip surface, at least one of a flat configuration, a curved configuration and combinations thereof.

8. The three jaw clamp apparatus as claimed in claim 1, wherein the jaws comprises at least one non-slip surface, at least one of a flat configuration, a curved configuration and combinations thereof.

9. The three jaw clamp apparatus as claimed in claim 1, wherein the jaws comprises at least one insert mounted on at least one contact surface.

10. The three jaw clamp apparatus as claimed in claim 1, wherein the jaw and the handle are made of a single member and one or more joint members.

11. The three jaw clamp apparatus as clamed in claim 1, wherein the structure of the handle of third member is configured to travel a minimum distance before coming in contact to the handle of first member upon complete opening of the jaw of the third member relative to the jaw of the first member.

12. The three jaw clamp apparatus claimed in claim 1, wherein an additional member placed between the handle of the first member and the handle of the third member is configured to reduce travel of the handle of the third member before coming in contact to the handle of the first member upon complete opening of the jaw of the third member relative to the jaw of the third member.

13. The three jaw clamp apparatus as claimed in claim 1, wherein the handles of the first, second and third member comprise structural gaps to permit the biasing unit(s) to pass through during their travel.

14. The three jaw clamp apparatus as claimed in claim 1, wherein the handles of the first, second and third member comprise structural gaps to permit cleaning agents and other accessory instruments to reach a space between the handles and biasing units.

15. The three jaw clamp apparatus as claimed in claim 1, wherein the handles of the first, second and third member comprise structural gaps to permit biasing units to pass through the structure of the handles of the members.

16. The three jaw clamp apparatus as claimed in claim 1, wherein members of biasing unit(s) are joined via welding at one point or surface, and handles of the first, second and third member comprise structural gaps to permit a welding electrode to reach the welding point or surface.

17. The three jaw clamp apparatus as claimed in claim 1, wherein the one or more biasing member(s) comprise a curved structure that reduces concentration of stress to promote longevity of the apparatus.

18. A three jaw clamp system for occluding a blood vessel and opening a lumen of the blood vessel comprising:
a pair of three jaw clamp apparatus, wherein each of the three jaw clamp apparatus, occludes the blood vessels; and
a support member connecting the pair of three jaw clamp apparatus, in proximity to each other, wherein each of the three jaw clamp apparatus, comprises:
a first member, a second member, and a third member; wherein each the support member comprises a handle and a jaw; and
wherein the first member and the second member being laterally moveable toward and away from one another;
wherein the third member being laterally moveable toward and away from the first member;
wherein the first member and the second member occlude the blood vessel in between corresponding jaws when a pinch force is applied on the handle of the first member and the handle of the second member;
wherein the third member and the first member occlude an adventitia of the blood vessel in between corresponding jaws when a pinch force is applied on the handle of the third member and the handle of the first member; and
wherein the third member and the first member maintain a tension in the adventitia by creating a holding tension on the adventitia to open the blood vessel,
wherein the jaw of the third member having width less than 60% of that of the jaw of the first member, and wherein the distance between tip of the jaw of the third member and a root of the adventitia is less than quarter of the perimeter of maximum size blood vessel to be occluded by the three jaw clamp system.

19. The three jaw clamp system as claimed in claim 11, wherein support member is one of a connector, an approximator, a retractor and a frame.

20. The three jaw clamp system as claimed in claim 11, wherein a biasing member placed in contact between each approximator and a second member is configured to impart adequate frictional force large enough to prevent undesired sliding and provide smooth motion upon applying deliberate force to cause relative motion.
